(19) United States
(12) Patent Application Publication
(10) Pub. No.: US 2005/0287078 A1
(43) Pub. Date: Dec. 29, 2005

(54) Minimizing powder retention on surfaces

(75) Inventors: Alf Niemi, Straengnaes (SE); Sven Calander, Strängnäs (SE); Lars Kax, Nykvarn (SE)

Correspondence Address: OBLON, SPIVAK, MCCLELLAND, MAIER & NEUSTADT, P.C., 1940 DUKE STREET, ALEXANDRIA, VA, 22314, US

(73) Assignee: MEDERIO AG, Hergiswil NW (CH)

(21) Appl. No.: 11/154,692

(22) Filed: Jun. 17, 2005

(30) Foreign Application Priority Data
Jun. 18, 2004 (SE) 0401612-7

(51) Int. Cl. A61L 9/04; A61K 9/14

(52) U.S. Cl. 424/46

(57) ABSTRACT

The invention relates to a method for improving the powder output, i.e. the emitted medication dose, from a dry powder inhaler device by minimizing the powder retention inside the device. Also therapeutic efficacy of the metered medication dose is hereby improved. It is found that adding a smaller amount of excipient than would be necessary in an ordered mixture, to a metered dose of an API formulation raises the emitted API dose when the dose is inhaled together with the excipient.

### REFERENCE TO PRIOR APPLICATIONS

This application claims the benefit of Swedish application SE 0401612-7 filed Jun. 18, 2004. U.S. application Ser. No. 10/898,372 filed Jul. 26, 2004, is incorporated herein in its entirety.

### TECHNICAL FIELD

The present invention relates to the minimization of powder retention on surfaces. In preferred embodiments the invention discloses a method and an arrangement for minimizing, for example in a dry powder inhaler device, retention of a metered dry powder medicament dose, thereby improving the yield in terms of emitted dose. In a highly preferred embodiment powder retention is minimized by adding an excipient dose to the medicament dose, whereby the excipient assists the release of the medicament dose during inhalation. In the invention smaller quantities of excipient are used compared to ordered mixtures according to prior art. In a further aspect the present invention relates to a method and a medical product for enclosing a metered, dry powder medication dose together with a dose of an excipient in a common dose container.

### BACKGROUND

Within health care today administration of medicaments by inhalation for distributing dry powder medicaments directly to the airways and lungs of a user is becoming more and more popular, because inhalation offers an efficient, fast, and user friendly delivery of the specific medication substance.

Dry powder inhalers (DPIs) have become accepted in the medical service, because they deliver an effective dose in a single inhalation, they are reliable, often quite small in size and easy to operate for a user. Two types are common, multi-dose dry powder inhalers and single dose dry powder inhalers. Multi-dose devices have the advantage that a quantity of medicament powder, enough for a large number of doses, is stored inside the inhaler and a dose is metered from the store shortly before it is supposed to be inhaled. Single dose inhalers use pre-metered doses and such inhalers are deposited with a limited number of individually packaged pre-metered doses, where each dose package or container is opened shortly before inhalation of the enclosed dose is supposed to take place.

Dry powder medicaments may be in a pure formulation consisting of an active pharmaceutical ingredient (API) only, or the formulation may comprise other substances for different purposes, e.g. enhancing agents for increasing the bio-availability and/or bio-activity of the API. Pharmacologically inert excipients may be included for diluting a potent API, in order to act as carrier of the API or to improve the flowability of the formulation to enhance metering and filling properties of the powder.

Powders with a particle size suitable for inhalation, i.e. particles in a range 0.5-5 μm, have a tendency of aggregating, in other words to form smaller or larger aggregates, which then have to be de-aggregated before the particles enter into the airways of the user. De-aggregation is defined as breaking up aggregated powder by introducing energy e.g. electrical, mechanical, pneumatic or aerodynamic energy. The aerodynamic diameter of a particle of any shape is defined as the diameter of a spherical particle having a density of 1 g/cm³ that has the same inertial properties in air as the particle of interest. If primary particles form aggregates, the aggregates will aerodynamically behave like one big particle in air.

The tendency to form aggregates of particles is aggravated in the presence of water and some powders are sensitive to very small amounts of water. Under the influence of moisture the formed aggregates require very high inputs of energy to break up in order to get the primary particles separated from each other. Another problem afflicting fine medication powders is electro-static charging of particles, which leads to difficulties in handling the powder during dose forming and packaging.

Methods of dose forming of powder formulations in prior art include conventional mass, gravimetric or volumetric metering and devices and machine equipment well known to the pharmaceutical industry for filling blister packs and gelatin capsules, for example. See WO 03/66437 A1, WO 03/66436 A1, WO 03/26965 A1, WO 02/44669 A1, DE 100 46 127 A1 and WO 97/41031 for examples of prior art in volumetric and/or mass methods and devices for producing metered doses of medicaments in powder form. Electrostatic forming methods may also be used, for example disclosed in U.S. Pat. No. 6,007,630 and U.S. Pat. No. 5,699,649.

Gelatin or plastic capsules and blisters made of aluminum or plastic, or laminates comprising aluminum and plastic foil are common prior art containers for metered single doses of dry powder medicaments. Typically, the user has to open the inhaler, insert at least one container into the inhaler, close it, push a button to force one or more sharp instrument(s) to penetrate a selected container, such that the dose may be accessed by streaming air when the user at leisure decides to inhale the dose. Besides a method of breaking the container open inside the inhaler and pour out the dose in a chamber first, the most common methods of opening the container are to punch one or more holes in the container itself or in a foil sealing the container or peel off the sealing foil. In the first case the powder is poured onto a surface inside the inhaler and made available for inhalation from there. In the second case the dose is aerosolized by inhalation air being forced through the container or the dose being shaken out of the container and immediately aerosolized by streaming air on the outside of the container.

Mixtures of API and excipients are very common in the pharmaceutical industry, particularly for tablets. Drug formulations containing API(s) and excipient(s) for making tablets need to be suitable in terms of potency, dosage mass, stability of form etc. Homogenous mixtures of powders comprising big, non-inhalable particles are standard in the industry. Such powder mixtures are easy to handle and make into tablets. However, tablets don't normally require that the particles of API and/or excipient ingredients are small enough to be inhalable. Therefore, dry powder preparations of so-called ordered mixtures of an API formulation of inhalable particles and an excipient formulation of larger particles, in some cases also including a small share of micronized excipient particles, are now common in prior art for inhalable medicaments. Common reasons for making ordered mixtures are e.g. to improve flowability of the powder mixture, to let the large excipient particles act as carriers for the API particles and to dilute a potent API formulation. Combining these effects is also a reason for making ordered mixtures. However, the ratio between API and excipient is limited if a stable, homogenous mixture is to be achieved, which in a filling process does not segregate small particles from big ones. The API formulation is limited to 4-5% by weight (w/w) of the mixture, higher blends gives problems. Therefore, the total dose mass of an ordered mixture, containing a therapeutically effective API dose, will often become too big for pulmonary delivery in a single inhalation.

Thus, there is still a need for improved efficacy of dry powder medicament doses in connection with an inhalation process for the release of a dose of medication powder by a DPI.

### SUMMARY

The present invention relates to the minimization of powder retention on surfaces.

The present invention relates in one embodiment to a method for improving the powder output, i.e. the emitted medication dose, from a dry powder inhaler device by minimizing the powder retention inside the device. The therapeutic efficacy of the metered medication dose is thereby also improved. Surprisingly, we have found that adding a smaller amount of excipient than would be necessary in an ordered mixture, to a metered dose of an API formulation, raises the emitted API dose when the dose is inhaled together with the excipient.

In a particular embodiment a dose of an excipient of approximately the same mass as a therapeutically effective API dose is filled together with the API dose in a common space of a dose container. Providing the doses are arranged such that the powders are aerosolized together upon inhalation, release of the API dose from the container is improved and retention of API particles in down stream inhaler channels is reduced, compared to if the excipient was not present. An inhalable API formulation, which is sticky and difficult to release and aerosolize by a DPI device, benefits from having a dose of an excipient of e.g. similar mass introduced for a joint delivery from the dose container. An advantage of the present invention is that the total mass of the accumulated doses is still small enough for an efficient delivery in a single inhalation from a DPI. If the same mass of API would be mixed with the excipient into an ordered mixture, the amount of excipient would have to be 20 times more. The total dose would then be too big for delivery by a single inhalation. The intended therapeutic effect would only be reached after multiple inhalations, if at all. Multiple inhalations put a strain on the user and increase the risk of incorrect administrations or non-compliance, whereby the intended therapy is jeopardized.

According to the invention, the improvement in emitted medication dose is not influenced by intentional or unintentional mixing of the doses of API and excipient after filling into the dose container, as long as the two doses are aerosolized together during inhalation. In fact, a somewhat random disorder of API and excipient particles can be an advantage in raising the emitted API dose figure. Any dry powder formulation for inhalation will benefit from the invention, such as pure API formulations or formulations comprising particles consisting of API and other ingredients and formulations of porous particles e.g. Technospheres® and microspheres. The method is particularly useful where the dry powder formulation in the medication dose is sticky and where particles of the formulation tend to attach themselves to surfaces with which they come in contact, such that they are difficult to set free. The present method may advantageously be applied to naturally sticky substances and formulations, but also to powders sensitive to ambient conditions such as elevated temperature and humidity.

The present invention also relates to a method for enclosing 1) a metered, dry powder medication dose comprising at least one active pharmaceutical ingredient (API) and 2) a metered, dry powder dose of at least one excipient in 3) a common space of a common dose container. The medication formulation is preferably a dry powder formulation adapted for inhalation and the excipient or excipients are preferably biologically acceptable dry powders that are in all respects compatible with the medication powder. The invention teaches that the respective formulations are metered and filled into a common space of the dose container. According to the invention, the deposited doses in the common dose container constitute a medical product. The method and the medical product are effective in raising the emitted medication dose output when the doses are delivered together by inhalation from a dry powder inhaler. The therapeutic efficacy of the metered medication dose is thereby improved.

In a further aspect of the invention the doses may be metered and deposited into a common aerosolization chamber inside a DPI from separate storage chambers or from separate receptacles inside the DPI in preparation for a delivery by inhalation. The addition of an excipient dose to a medication dose at the inhalation stage improves the release of the API of the medication, such that the emitted medication dose increases and the retention in the aerosolization chamber and in the down stream airflow channels decreases. Preferably, the two doses are arranged to be aerosolized together, simultaneously, e.g. by partly mixing the doses in the dose container before they are inhaled. The excipient dose mass is not critical in order to achieve an improved quality and quantity of the emitted API dose.

### DESCRIPTION OF THE DRAWINGS

The invention will be described in the form of a preferred and illustrative embodiment and by means of the attached drawings, wherein like reference numbers indicate like or corresponding elements and wherein:

FIG. 1 illustrates in perspective (FIG. 1a), top (FIG. 1b) and side (FIG. 1c) views a particular embodiment of a sealed dose container filled with a dose of a medicament and a dose of an excipient;

FIG. 2 illustrates a sealed dose container filled with a dose of a medicament consisting of two deposits and a dose of an excipient consisting of three deposits;

FIG. 3 illustrates a sealed dose container after agitation filled with a dose of a medicament consisting of two deposits and a dose of an excipient consisting of three deposits where the doses have become partly mixed;

FIG. 4 illustrates in a graph results of a climate test showing the drop in fine particle dose, FPD, of Atrovent® with active substance being ipratropium bromide.

FIG. 5 illustrates in a flow diagram the steps of the invention for joining a metered medication dose and an excipient dose in a common dose container.

### DESCRIPTION OF PREFERRED EMBODIMENTS

The present invention relates in a preferred embodiment to a method for improving the powder output, i.e. the emitted medication dose, from a dry powder inhaler device by minimizing the powder retention inside the device. The addition of an excipient dose to a medication dose helps to release the medication dose, i.e. the API, and entrain it into inspiration air when the doses are inhaled together and delivered to a user of a DPI device. Retention of medication particles inside the DPI is much reduced, not only in the dose carrier, but also in the downstream air channels, which direct the airstream carrying the aerosolized medicament dose out of the DPI and into the users air ways. Retention in the DPI may result in powder build-ups and may affect the efficacy of the inhaler adversely. Further, built-up medicament may come loose during an inhalation, which may result in an overdose to the user.

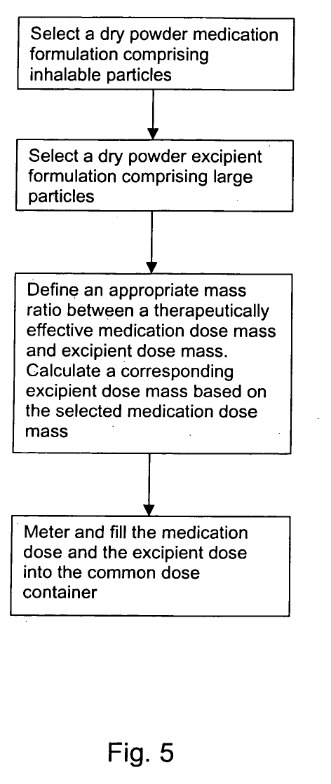

The present invention also teaches a method, illustrated in a flow diagram in FIG. 5, for joining a metered, dry powder medication dose and a dry powder dose of a biologically acceptable excipient in a common dose container. Surprisingly, we have found that a medical product, based on the present method, improves the emitted dose, i.e. the output mass of the active ingredient of the medication powder dose, when the joined doses are inhaled together from a dry powder inhaler device.

Surprisingly, we have found that the present invention can advantageously be applied to many types of dry powder medicament formulations. Examples of medical dry powders particularly suitable for the present method include formulations comprising proteins, including peptides, lipids, water-soluble excipients or APIs, powders of porous particles, e.g. Technospheres® and microspheres. An inhalable API formulation, which otherwise would be pre-mixed with an excipient into an ordered mixture with the object of attaining a high degree of efficacy when delivered by inhalation, can reach the same or even better efficacy and low retention by applying the teachings of the present invention instead. The addition of the excipient dose acts as a cleaning agent and helps to release the medication dose and entrain it into inspiration air when the doses are inhaled by use of a dry powder inhaler device. The present invention also offers added benefits by using only a fraction of the mass of the excipient of an ordered mixture in order to deliver a therapeutically effective API dose. The total mass of a therapeutically effective dose of an ordered mixture is often too big to be suitable for a single act of inhalation. A high metered dose mass may not become completely aerosolized by the DPI and too much of the metered dose is then left unreleased in the dose carrier after an inhalation. The big amount of excipient in the mixed dose may cause problems for the user during the inhalation and may trigger coughing spells. The cumbersome step of producing the ordered mixture is further made redundant by the present invention, when put to use.

In a further aspect, it is well known in the art that many important medicaments in dry powder formulations are sensitive to high levels of humidity, such that the emitted particle dose out of an inhaler device drops drastically as the relative humidity in the air increases. This sensitivity to ambient conditions is especially noticeable among the new protein-based, inhalable medicaments currently in development or recently introduced into the marketplace, e.g. medicaments directed towards treatment of systemic disorders. In recent years, the pharma industry, having interests in inhalable medicaments, has directed most development resources to the formulation side of product development and the delivery systems, i.e. dose packaging and the inhaler devices, have been less in focus. Thus, the teachings herein concern improvements in drug delivery performance by inhalation.

A highly preferred formulation of an API for inhalation is preferably chemically and biologically stable under storage and in-use conditions, and has a high bio-availability and bio-activity, a suitability for a filling process and a narrow particle size distribution. There are a number of well-known techniques for obtaining a suitable primary particle size distribution that will ensure correct lung deposition for a high percentage of the dose mass. Such techniques include jet-milling, spray-drying and super-critical crystallization. There are also a number of well-known techniques for modifying the forces between the particles and thereby obtaining a powder with suitable adhesive forces. Such methods include modification of the shape and surface properties of the particles, e.g. porous particles and controlled forming of powder pellets, as well as addition of an inert carrier with a larger average particle size (so called ordered mixture). A simpler method of producing a finely divided powder is milling, which produces crystalline particles, while spray-drying etc produces generally more amorphous particles. Novel drugs, both for local and systemic delivery, often include biological macromolecules, which present completely new demands on the formulation and the production process. Examples of problems, which need to be addressed when developing a formulation for inhalation comprising an API and optionally other substances, are:
API stability
Absorption of the API in the lung
Solubility of the API
Particle size distribution
Dilution of API potency
Elimination of unpleasant taste
Powder flowability

When a working formulation of an API has been developed and regulatorily approved together with a chosen packaging and dose delivery system, the threshold of improving the formulation chemically or biologically is very high, because the whole regulatory process must be repeated. Besides the time and cost involved in developing a new formulation, most time and money will be spent on regulatory work. From this aspect, the present invention may provide a fast road to higher medical efficacy by making a switch to a different technical platform possible. Technically, it is very straightforward to implement the present invention and to switch the packaging and dose delivery systems. A new dose container may be developed or an existing one may be chosen capable of accepting a dose of the original API formulation and a dose of a selected excipient, such that the doses will be aerosolized simultaneously when made available in a DPI. Examples of suitable DPIs, which may be used with the present invention are described in our publications U.S. Pat. No. 6,622,723 and U.S. Pat. No. 6,422,236. Regulatorily, combining a well-known, proven formulation with a biologically acceptable excipient does not require extensive development and clinical studies to aquire an approval. The regulatory process is normally in such cases uncomplicated and quick in comparison.

Dose Forming

An advantage of the present invention is that prior art methods of dose forming of medication and excipient powder formulations for inhalation are easily applied, such as conventional mass, gravimetric or volumetric dose metering. Filling devices and machine equipment well known to the pharmaceutical industry for filling blister packs and gelatin capsules, for example, may be used. Electrostatic forming methods may also be used, for example as disclosed in our publication WO 02/11803 (U.S. Pat. No. 6,696,090) disclosing a method and a process of preparing a so called electro-powder, suitable for forming doses by an electro-dynamic method, further described in our publication U.S. Pat. No. 6,868,853, which both are incorporated hereby in this document in their entirety by reference. These disclosures stress the importance of controlling the electrical properties of a medication powder and points to the problem of moisture in the powder and the need of low relative humidity in the atmosphere during dose forming. Ambient conditions during dose metering, filling and container sealing should be closely controlled. The ambient temperature is preferably limited to 25° C. maximum and relative humidity preferably limited to 15% Rh maximum, but the actual permissible relative humidity depends on the specific formulation and some cases may require much less than 15% Rh, even less than 5% Rh. The powder formulation is also to be kept as dry as possible during the dose forming process. Further, it is very important to control the electric properties of the powders and to apply electric charging and discharging as needed, regardless of which method of dose forming is to be used. Fine powders pick up static electric charges extremely easily, which can be advantageously used in dose forming, if the charging and discharging is under proper control. Keeping the ambient relative humidity low ensures that only a very small, acceptable amount of water is enclosed in the dose container together with the dose and not enough to present a threat to the stability of a moisture sensitive substance and the fine particle dose (FPD) of the dose.

Disregarding which filling method or combination of methods is preferred, in another embodiment of the invention, a dose comprising at least one API and a dose comprising at least one biologically acceptable excipient are separately metered, whereupon the metered doses are filled together into a dose container, optionally first being at least partly mixed prior to filling. In a further particular embodiment, demonstrating the versatility of the invention in a non-limiting example, a dose of excipient is filled into the container in a first step, but not spread out inside. Then, in a second step the medicament is filled into the container, optionally on top of the excipient. Optionally, a further amount of excipient is deposited on top of the dose. A further option is to agitate by e.g. shaking or vibrating the dose container after filling and optionally after sealing of the container, whereby the excipient(s) become roughly mixed with the medicament powder.

Preferably, the de-aggregating system should be as insensitive as possible to variations in the inhalation effort produced by the user, such that the delivered aerodynamic particle size distribution in the inhaled air is largely independent of the inhalation effort over a certain minimum level. A very high degree of de-aggregation is generally obtained when one pays attention to the following:
a suitable formulation of the powder (particle size distribution, particle shape, adhesive forces, density, etc)
a suitably formed dose of the powder adapted to the capabilities of a selected inhaler device
an inhaler device providing shear forces of sufficient strength in the dose to release and de-aggregate the powder (e.g. turbulence)

A method and a device for de-aggregating a powder are disclosed in our U.S. Pat. Nos. 6,840,239 B1 and 6,892,727 B1, the teachings of which are included in this document by reference.

Optimized Dose Delivery

Suitable dose sizes for inhalation are typically in a total mass range from 1 mg to 20 mg. Smaller doses than 1 mg are difficult to meter and fill consistently and doses having a mass exceeding 20 mg may be difficult to release and de-aggregate completely in a DPI. Many of the new protein-based active substances require a metered mass of the API in the order of 1-5 mg to give the desired therapeutic effect when inhaled. If the medicament comprising the API is a candidate for being included in a mixture, further comprising an excipient of bigger particles, typically of average size between 20 and 200 μm, acting as carriers of the medicament, one must keep in mind that a stable, homogenous, ordered mixture in bulk quantity that does not begin to segregate when used in a repetitive filling operation, generally cannot hold more than 4-5% w/w of the medicament. Segregation means that small drug particles separate from the big excipient ones, leading to different concentrations of the API in different parts of the bulk powder store. Given that the medicament mass is in the range 1-5 mg, i.e. pure API having a therapeutic effect, a metered dose of an ordered mixture will be in a range from 20 to 125 mg. A dose mass in this range is generally not suitable for inhalation. APIs for systemic absorption by pulmonary delivery should have aerodynamically very small particles in a range 1-3 μm, which makes it difficult to make a homogenous, ordered mixture, which does not segregate when later used in a filling process. If the concentration of API varies in the bulk powder mixture and if segregation occurs during handling and in the filling process, it will be impossible to know how much API drug is filled each time. A particular aspect of the present invention presents a solution to this problem by using far less excipient, not in an ordered mixture with the medicament as in prior art, but separately dosed into the same dose container or aerosolizing chamber as the medicament dose. Paradoxically, this method is unknown in prior art. Perhaps the answer to the paradox is that the focus in the pharmaceutical industry has been on developing and producing stable drug formulations, not intended for inhalation, which were easy to mix, meter and fill industrially using standard methods and equipment. It is only recently that pharmaceutical companies have started to look at inhalation as a new method of administration, and have come to realize that inhalation of drugs means completely new and different demands on drug formulations.

The present invention generally uses mass (weight) ratios API/excipient in a range 1/20-20/1, including endpoints, and all values and subranges therewithin, for example 1/10-10/1, 1/5-5/1, 1/18, 1/17, 16/1, 12/1, etc. In other words, the excipient dose mass, according to the disclosure, ranges from 5% to 95% of the total mass of API and excipient doses taken together, including for example 10%, 15%, 20%, 25%, 30%, 40%, 50%, 60%, 70%, 80%, 90%, 92%, 93%, 94% and less than 95%. In contrast, in ordered mixtures typical values are API/excipient in a range from 1/100 to 5/100, in other words the excipient's share of the total dose mass of an ordered mixture is typically in a range from 95% to 99%. In fact, the present invention simplifies the dose filling in many cases, because the complex process of making a stable mixture of the API formulation and a suitable excipient is eliminated. For example, a first dose of the formulation containing the API is metered and filled into a dose container or aerosolizing chamber and a second dose of at least one excipient is also filled into the same space as the first dose in the dose container or chamber. The order of filling the first and the second doses makes no difference for the invention, and filling may occur simultaneously as well. In a particular embodiment each of the respective doses may comprise more than one powder deposit. Provided the first and second doses are released simultaneously together upon inhalation, the emitted dose of the API will be boosted, compared to not having the excipient dose present in the dose container. In a particular embodiment the excipient and the API doses have been mixed to some extent, for example by being agitated, e.g. by vibrating them or by giving them a physical shock in the container prior to inhalation. An advantage of the invention is that the road to regulatory approval may be considerably shorter compared to taking a new formulation through the necessary, regulatory steps. A further advantage of the disclosure is that metering and filling of the medicament dose may become simpler compared to filling an ordered mixture. Both the cleaning excipient and the medicament are often easy to meter separately.

Surprisingly, we have found that even using a small amount of excipient powder, which is used to coat the inner surfaces of the container or aerosolization chamber prior to filling of the API dose, may be sufficient to raise the emitted dose figure of the API and to reduce the retention of the API inside an inhaler device. From a filling point of view, however, it is preferred to fill the container with a first and a second dose of the medicament and the excipient respectively, in any order or simultaneously, where each metered dose consists of at least one deposit of the respective dry powder formulation. Later, when the dose container is opened and the doses aerosolized during an inhalation, the particles of the excipient dose act as cleaning agents for the container and the internal parts of the inhaler, whereby a high share of the medication powder particles that stick to the interior surfaces before and during the inhalation are forcibly released, probably by impaction, and entrained in the streaming inhalation air. The cleansing effect is very obvious whether or not the medication dose has been agitated or mixed with the excipient dose after filling but prior to an inhalation, provided the doses are released simultaneously together. A possible explanation for this insensitivity of the invention to the extent of mixing of the powders in the dose container not limiting of the invention is that when the doses are released generally simultaneously, large particles act partly as carriers by picking up small particles and partly as blasting grit hitting the interior walls of the dose container and the air channels, whereby many small particles are released into the airstream. Retention of small medicament particles is thus reduced.

Generally, dry powder medicament doses need to be protected by an enclosure not only during storage, but also when inserted in an inhaler, e.g. a single dose DPI, where the dose and its enclosure are kept in a ready state before delivery in an inhalation at a point in time decided by the user. New types of dry powder medicaments, not least for systemic treatment, have a rather short expiry date and they are generally quite sensitive to ambient conditions, especially moisture during storage and in use. Hence, the demands put on dose protection and inhaler devices in handling sensitive doses are therefore much higher than for prior art devices as used e.g. for administering traditional medicaments against respiratory disorders.

In the development of new and improved types of single dose dry powder inhalers, see our U.S. Pat. Nos. 6,622,723, 6,422,236, 6,868,853, 6,571,793 and 6,840,239, we have also developed dose filling methods, see our U.S. Pat. No. 6,592,930 and WO 04/110539, all of which are incorporated in this document in their entirety by reference. In the development work, particular attention has been devoted to sticky substances per se. Such dry powders are inclined to leave a high percentage of the active substance, the active pharmaceutical ingredient(s), retained on the inner surfaces of the dose container or aerosolization chamber and on the internal walls of the air channels inside the inhaler device, through which the airflow passes carrying the released dose into the airways of the user. Not surprisingly, if the humidity of the surrounding air is high, we've found that medical powders in general are inclined to stick to any surface with which they come in contact. What degree of air humidity is deemed to be high depends on the sensitivity to humidity of the powder. The retention effect for a sticky powder depends, inter alia, on the structure of surfaces in contact with the powder, the surface areas, the materials concerned, the design of the inhaler device and the aerosolization and de-aggregation forces provided by the inhaler, to name a few important factors. Time is another factor e.g. when stickiness is due to high moisture. Different powders adsorb more or less humidity and at different rates. However, many medical powders are affected within milliseconds of being exposed to humidity in the ambient air. Other powders adsorb water more slowly. In any case, it is not satisfactory having an inhaler system designed such that the user may open a dose container first, allowing the ambient atmosphere access to the dose therein for an undefined time period of seconds or even minutes before an inhalation commences.

In a preferred embodiment of the invention, doses of the respective formulations of active substance, API, and excipient are separately metered and filled into the same dose container, where the doses, intentionally or unintentionally, may or may not be mixed after filling. However, a nonuniform, random mixture, if created by shaking for instance, is characterized in that it does not constitute an ordered mixture, but a nonuniform mixture may be an optional method of attaining a joint and simultaneous release of the doses when inhaled. Naturally, the excipient or excipients must be compatible in all respects with the medication powder. The improvement in emitted API dose, which follows upon inhalation of both doses together, as a percentage of the metered API dose is very significant and the improvement corresponds to a powerful reduction in retention. Particularly sticky powders may benefit from the present invention, because the relative improvement in emitted dose may be more important than for more easily aerosolized powders. Powders may be naturally sticky or conditionally sticky or both, e.g. if affected by humidity. A particular embodiment requires that at least one deposit of the medicament is deposited in the dose container and that a deposit of the excipient dose is deposited on each diametrically opposed side of the at least one medication deposit. The respective deposits of the medicament and the excipient are preferably of approximately the same mass and the respective deposits added together constitute the respective medicament and excipient doses. Typically, the dose mass of the excipient is roughly the same as the mass of the medication dose, but other mass ratios may be used. The optimal deposition pattern of the doses in the dose container, e.g. if doses are split up in several deposited deposits, depends on how the DPI aerosolizes the powder in the dose container. In any case, the excipient dose is to be aerosolized together with the medication dose, but a release pattern of alternating release of parts of the medication dose interleaved with release of parts of the excipient dose is equally possible in order to fully realize the cleaning effect of the excipient in the course of an inhalation taking place. The coarse excipient particles may act similarly to a sandblasting device, i.e. to physically set medicament particles free by sheer impaction power, but coarse particles also tend to collect small particles and carry them into the airstream, where the small particles are released by turbulent forces.

The excipient may comprise fine particles having sizes ≦10 μm, particles ≧10 μm or the excipient may comprise fine particles <10 μm and coarse particles >10 μm. The excipient particles having an aerodynamic diameter (AD) of 10 μm or more are deposited by impaction in the mouth, throat and upper airways upon inhalation, because the mass of these excipient particles is generally too big to follow the inspiration air into the lung. Therefore, excipients are selected inter alia with consideration for using substances that are harmless when deposited in the areas concerned. However, an excipient formulation may comprise more than one excipient. For instance it is often advantageous to include an excipient containing small particles ≦10 μm in a mixture with an excipient containing big particles ≧10 μm, more typically ≧25 μm. This mixture flows easily and a metered dose of the mixture holds together when lightly compacted and makes the filling process simple. The mass ratio between small particles and big ones can be, e.g., in a range 0.01-0.1 and typically 0.02-0.05 for best operation. The excipients used may or may not be of the same substance.

Suitable excipients for inclusion in a dose container or chamber include monosaccarides, disaccarides, polylactides, oligo- and polysaccarides, polyalcohols, polymers, salts or mixtures from these groups, e.g. glucose, arabinose, lactose, lactose monohydrate, lactose anhydrous [i.e., no crystalline water present in lactose molecule], saccharose, maltose, dextrane, sorbitol, mannitol, xylitol, sodium chloride, calcium carbonate. A particular excipient is lactose. Lactose in a dry powder form, so called Respitose® from DMV International having 95% of particles larger than 32 μm, has been successfully used as a cleaning excipient in many inhalation experiments of ours.

The moisture properties of any proposed excipient should be checked before it is chosen to be used as a cleaning agent. If an excipient gives off water, after dose forming, it may negatively affect the API in the medicament dose, such that the fine particle dose, FPD, deteriorates rapidly after sealing of the dose container. Therefore, excipients to be in contact with or mixed with the medicament are preferably to be selected among acceptable excipients, which have good moisture properties in the sense that the excipient will not adversely affect the FPD of the API(s) for the shelf life of the product, regardless of normal changes in ambient conditions during transportation and storage. Suitable “dry” excipients are to be found in the above-mentioned groups. In a particular embodiment of the present invention, lactose is selected as the preferred dry excipient and preferably lactose monohydrate. A reason for selecting lactose as excipient, is its inherent property of having a low and constant water sorption isotherm. Excipients having a similar or lower sorption isotherm can also be considered for use, provided other required qualities are met.

The disclosed method counteracts as far as possible any adverse influence that e.g. humidity in the air may have on the fine particles in the dose. Minimizing the dose exposure to the atmosphere may preferably be done by implementing a breath actuation mechanism coupled to opening of the dose container in the inhaler. But the present invention may be advantageously used to boost performance from a dry powder inhaler device. Examples of problems in prior art dry powder inhaler devices, having negative effects on the emitted dose are:
Inhaler design provides too low airflow turbulence close to dose during inhalation;
Selected dose container is bigger than necessary and provides too much internal surface area for powder to stick to;
Inhaler and dose container present high sticking effects between dose particles and internal surfaces of dose container and inhaler;
User interface of the inhaler gives ambient humid air access to the dose for a long time before an inhalation actually takes place;
   Such failings in prior art DPI devices may be rendered less detrimental and the emitted dose improved by the adoption of the present invention. Most preferably the present invention is applied in an inhaler incorporating an Air-razor device for a gradual dose release in a prolonged dose delivery period, as described in our U.S. Pat. No. 6,840,239.

### EXAMPLE 1

### Mixtures of API and Excipient

In the course of developing methods and products according to the present invention different APIs were mixed with excipients in different ratios. The objectives were to find inter alia suitable formulations and methods of filling doses, but also to optimize inhaler techniques and interaction between inhaler and dose container. Table 1 below discloses some examples of volumetrically dosed mixtures API/excipient and the resulting emitted dose when delivered by a DPI.

Conclusions

As shown in Table 1, mixtures with far more micronized API (insulin) than 5% were used in these tests. The objective was to find suitable excipients and to see how different mixing ratios affected the emitted dose. The mixtures were produced under laboratory conditions in small quantities and remained quasi-stable under the run of tests. The emitted dose as percentage of total recovered dose was measured and the results were used in the development of the current invention.

TABLE 1

| | | | Emitted | Retention |
|---|---|---|---|---|
| | | | Dose in % of | in % of |
| | | Ratio API/ | recovered | recovered |
| API | Excipient | Excipient | dose | dose |
| Micronized | Mannitole | 50/50 | 83 | 17 |
| insulin | | | | |
| Micronized | Respitose ® | 20/80 | 93 | 7 |
| insulin | | | | |
| Fluticasone | Respitose ® + 10% | 1/44 | 88 | 12 |
| | micronized lactose | | | |

### EXAMPLE 2

### Ipratropium Climate Stability Tests

This test was made in order to find out how sensitive ipratropium bromide is to moisture. Commercially available Atrovent® capsules containing ipratropium bromide and excipient were bought in from our local pharmacy and introduced into the laboratory together with the HandiHaler® dry powder inhaler device. The powder was withdrawn from the originator's capsules and transferred to the capsules again after climate storage. The aerodynamic fine particle fraction (FPF) in the emitted dose from the HandiHaler® was measured using Andersen impactors according to European Pharmacopoeia (EP) and US Pharmacopoeia (USP). All analytical work were then performed according to standardized methods using a state of the art High Performance Liquid Chromatograph (HPLC) system.

Test S1

Aerodynamic fine particle fraction of metered and delivered dose out of Handihaler® using Atrovent® formulation powder was analyzed. Transfer of powder from and back into originator capsules was performed in relative humidity below 10%. The test was performed with 4 kPa pressure drop over the HandiHaler® at room temperature and laboratory ambient conditions.

Test S2

An in-use stability test was carried out of the aerodynamic fine particle fraction of metered and delivered dose out of Handihaler®. From the blister holding the Atrovent® capsules the powder was transferred to a medium moisture barrier container and sealed. The containers were put for 1 month in 25° C. and 60% Rh. The container holding the powder was then put in an exicator for 2 h before tests were performed. The inhaler test was performed with 4 kPa pressure drop over the HandiHaler® at room temperature and laboratory ambient conditions.

Test S3

The same test as in S2 was carried out except that the containers were put for 1 month in 40° C. and 75% Rh.

Conclusions

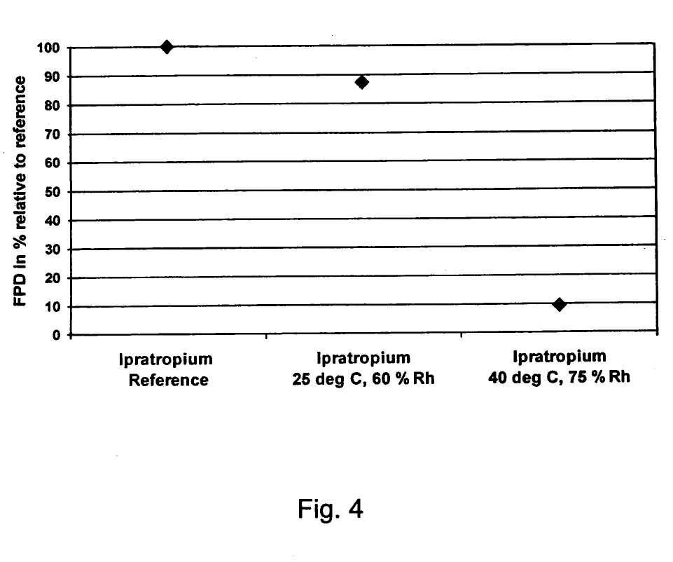

It is obvious from the graph in FIG. 4 showing the drop in fine particle dose, FPD, that ipratropium bromide is a very moisture sensitive substance.

### EXAMPLE 3

In order to illustrate the positive effect on emitted dose, i.e. the mass of the medicament powder entrained in inspiration air leaving a typical DPI, the following tests were carried out in our laboratory.

A pure, micronized, recombinant, human insulin in dry powder form was selected as the medicament test substance. Lactose in a dry powder form, so called Respitose® from DMV International having 95% of particles larger than 32 μm, was selected as a cleaning excipient.

Four dose containers, aluminum blisters constituting so called pods, were filled in a dry climate with nominally 2.5 mg insulin. The filled containers were designated ‘A’.

Four further containers, identical to the first four, were filled in the same, dry climate with nominally 2.5 mg insulin and 2.5 mg Respitose®, in separate filling steps, making a total dose of 5 mg and a mass ratio of 50/50 between insulin and Respitose®. The filled containers were designated ‘B’.

The containers were adapted for insertion into a single dose DPI.

Two containers of type ‘A’ and two containers of type ‘B’ were put for an hour before testing in a climate cabinet set at room temperature and approximately 90% relative humidity and the remaining containers were stored in the laboratory under normal ambient conditions.

TABLE 2

| | | | | |
|---|---|---|---|---|
| | Emitted Dose in | Retention | | |
| | % of metered | in % of | Emitted | Retention, |
| Type of filling | dose | metered dose | dose, μg | μg |

| | | | | |
|---|---|---|---|---|
| Ambient ‘A’ | 87 | 13 | 2166 | 320 |
| Ambient ‘B’ | 93 | 7 | 2296 | 185 |
| Humid ‘A’ | 76 | 24 | 1982 | 610 |
| Humid ‘B’ | 79 | 21 | 1992 | 543 |

The emitted doses were measured using a total of four DPIs, two per type of filling (‘A’ and ‘B’) and climate. Emitted dose was measured using a HPLC analyzer. Retention in the containers and in the suction tube and mouthpiece of the inhalers were also measured using the HPLC analyzer. Results are presented in Table 2 above.

Conclusions

The tests show conclusively that the cleaning excipient Respitos boosts the emitted dose (ED) leaving the inhaler. This is especially noticable under ambient conditions where the ED increases from 87 to 93% of the metered dose and retention is reduced by almost 50% from 13 to 7%. In the humid case the improvement is significant by 3 percent units, retention is reduced by approximately 15%, from 24 to 21%. For comparison, an ordered mixture containing 2.5 mg of insulin API would have a total mass of at least 50 mg and thus not be suitable for inhalation. It is also interesting to note that the efficacy of the test DPI system is very high even under extremely humid conditions.

The disclosed method is easily adapted to the particular type of dose container, which has been selected for insertion into a particular, adapted dry powder inhaler. As already pointed out, different types of dose containers are advantageously used in the present invention. Examples of containers are aluminum or plastic single dose blisters of varying size and design and also capsules of gelatin, cellulose or plastics. Prior art blister packages for dry powder medicaments, intended for inhaler use, often have a fairly thin polymeric seal, which can be easily ripped or punched open before the dose is supposed to be inhaled. Another common seal is a peelable foil such that the blister is peeled open prior to inhalation of the enclosed dose. Yet another type of prior art dose container is the capsule. Capsules are often made of gelatin, but polymers and cellulose and other materials are also used. A common problem for prior art blisters and capsules used for dry powder doses for inhalation is that the primary package does not protect sensitive substances from moisture well enough during storage and in use.

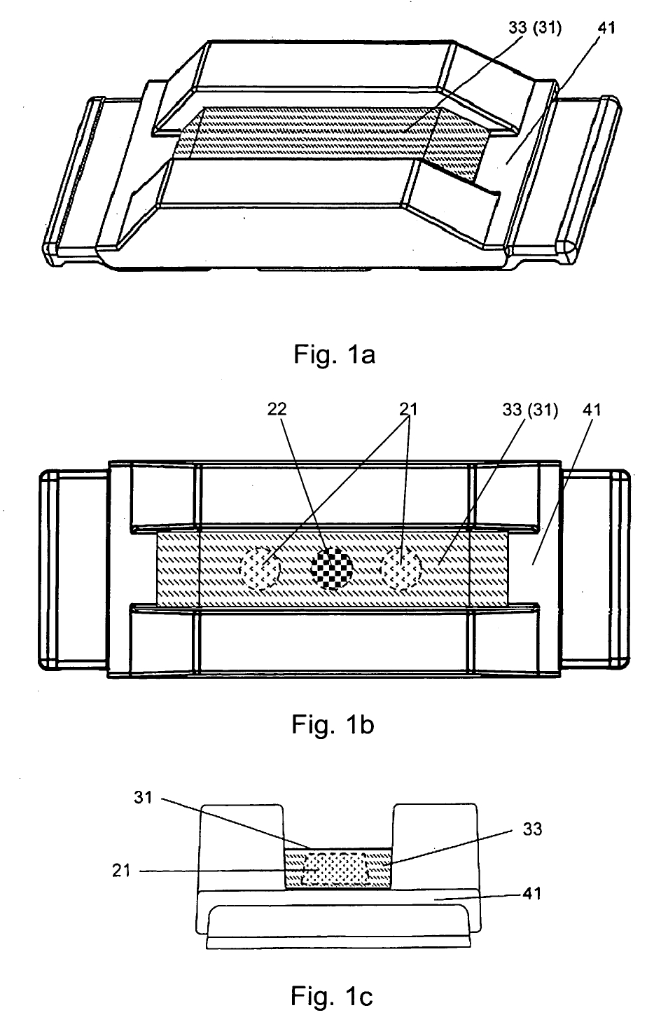

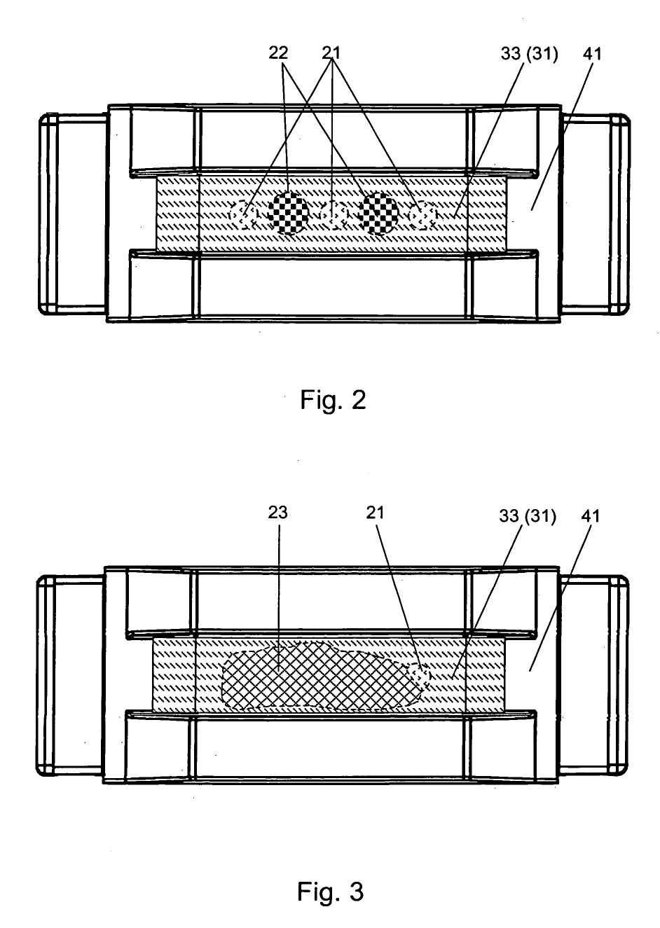

Using a new type of blister pack, a so-called pod (patent pending), as a particular embodiment of a sealed dose container, is to be preferred in an application where the present invention is to be put to use. A pod container may be made as a high barrier seal container offering a high level of moisture protection and which is in itself dry, i.e. it does not contain water. See FIG. 1 illustrating a pod carrying a sealed container in a perspective drawing. FIG. 1a shows a sealed dose container 33 (seal 31) put into a protective casing 41 adapted for insertion into a dry powder inhaler. FIG. 1b shows a top view of the carrier/container and indicates a dose of a dry powder medicament 22 and a dose of a dry powder excipient consisting of two deposits 21 inside the container 33 under a seal 31. FIG. 1c illustrates a side view of the carrier/container in FIG. 1b. FIG. 2 illustrates a similar container to FIG. 1, but the medicament dose consists of two deposits 22 and the excipient dose consists of three deposits 21. FIG. 3 illustrates the dose container in FIG. 2 after agitation of the container, whereby the deposits 21 and 22 have become partly mixed in a deposit 23.

In a further aspect of the invention a dry powder medicament dose, comprising at least one API, and a dry powder excipient dose, comprising at least one excipient, may be metered and deposited together into a common aerosolization chamber in a DPI from separate storage chambers or from separate receptacles inside the DPI in preparation for delivery by inhalation. The invention teaches that the addition of an excipient dose to a medication dose at the inhalation stage improves the release of the API of the medication powder dose, such that the emitted API dose increases and the retention in the aerosolization chamber and in the down stream airflow channels decreases, compared to if the excipient dose was not present. The therapeutic efficacy of the metered medication dose is hereby improved. It is not necessary to arrange a mixing of the doses, as long as the excipient dose generally is aerosolized simultaneously with the medication dose. The excipient dose mass is not critical to achieve an improvement in the quantity of the emitted API dose. The big excipient particles will impact and stick in the mouth and throat and become swallowed and will have no detrimental effect on the efficacy of the emitted dose.

Preferred embodiments according to the invention include:

1. A method of improving an emitted dose of a metered dry powder medicament dose comprising at least one active pharmaceutical ingredient from a dry powder inhaler device, comprising:
placing a metered dose of at least one biologically acceptable excipient and the metered medicament dose in a common space of a dose container or internal aerosolization chamber in a manner such that the dose of the medicament and the dose of the excipient are aerosolized together by the inhaler device during an inhalation, whereby particles of the excipient dose set particles of the medicament dose free into a stream of inhalation air, thereby raising an emitted medicament dose mass and reducing retention of medicament particles inside the inhaler device, whereby the yield of the medicament dose increases due to the presence of the excipient dose as compared to both the absence of the excipient dose and the presence of the excipient dose in an amount of less than 20:1 wt/wt excipient/medicament, i.e. excipient's share of medicament and excipient doses taken together is less than 95%.

2. A composition comprising a metered dose of at least one biologically acceptable excipient and a metered dry powder medicament dose, both contained within a common space of a dose container or internal aerosolization chamber of a dry powder inhaler device in a manner such that the dose of the medicament and the dose of the excipient can be aerosolized together by the inhaler device during an inhalation, whereby particles of the excipient dose set particles of the medicament dose free into a stream of inhalation air, thereby raising an emitted medicament dose mass and reducing retention of medicament particles inside the inhaler device, whereby the yield of the medicament dose increases due to the presence of the excipient dose as compared to both the absence of the excipient dose and the presence of the excipient dose in an amount of less than 20:1 wt/wt excipient/medicament, i.e. excipient's share of medicament and excipient doses taken together is less than 95%.

3. A method for delivering a dose of a metered dry powder medicament comprising at least one active pharmaceutical ingredient from a dry powder inhaler device, comprising:
inhaling a metered dose of at least one biologically acceptable excipient and the metered medicament dose from a common space of a dose container or internal aerosolization chamber in a manner such that the dose of the medicament and the dose of the excipient are aerosolized together, whereby particles of the excipient dose set particles of the medicament dose free into a stream of inhalation air, thereby raising an emitted medicament dose mass and reducing retention of medicament particles inside the inhaler device, whereby the yield of the medicament dose increases due to the presence of the excipient dose as compared to both the absence of the excipient dose and the presence of the excipient dose in an amount of less than 20:1 wt/wt excipient/medicament, i.e. excipient's share of medicament and excipient doses taken together is less than 95%., with optional mixing of the excipient and medicament before and/or during inhalation.

4. The method or composition according to the invention, where the mass ratio between the medication dose and the excipient dose is in a range 1:20-20:1.

5. The method or composition according to the invention, where the dry powder medicament comprises, consists essentially of, or consists of inhalable powder particles having a mass median aerodynamic diameter within a range from about 0.5 μm to about 5 μm.

6. The method or composition according to the invention, where the excipient is at least one from a group consisting of monosaccarides, disaccarides, polylactides, oligo- and polysaccarides, polyalcohols, polymers, salts and mixtures thereof.

7. The method or composition according to the invention, where the excipient comprises large particles to at least 90% by mass.

8. The method or composition according to the invention, where the excipient comprises large particles bigger than 20 μm in size to at least 90% by mass.

9. The method according to the invention, comprising the further step of agitating the dose container enclosing the metered doses using electrical or mechanical energy such that the doses inside the container become at least partly mixed, and the mixed composition produced.

10. The method or composition according to the invention, where the mass ratio between the medication dose and the excipient dose is in a range 1:10-10:1.

11. The method or composition according to the invention, where the dry powder medicament comprises, consists essentially of, or consists of inhalable powder particles having a mass (weight) median aerodynamic diameter within a range from about 0.5 μm to about 5 μm.

12. The method according to embodiment 2, comprising the further step of
selecting the at least one excipient from a group consisting of monosaccarides, disaccarides, polylactides, oligo- and polysaccarides, polyalcohols, polymers, salts or mixtures thereof.

13. An arrangement in a dry powder inhaler device for improving emitted dose of a metered dry powder medicament dose, comprising at least one active pharmaceutical ingredient, when said metered dose is delivered, wherein
a separately metered dose of at least one biologically acceptable excipient formulation is included with the metered medicament dose in a common space of a dose container or internal aerosolization chamber, said excipient dose having a pre-determined ratio of mass relative the metered medicament dose;
the dose of the medicament and the dose of the excipient are arranged in the dose container such that said doses become aerosolized together by the inhaler device during an inhalation, and
whereby particles of the excipient dose set particles of the medicament dose free into a stream of inhalation air, thereby raising an emitted medicament dose mass and reducing retention of medicament particles inside the inhaler device, whereby the yield of the medicament dose increases due to the presence of the excipient dose in the dose container.

14. The arrangement according to the invention, wherein
the dose container enclosing the metered doses is agitated using electrical or mechanical energy such that the doses inside the container become at least partly mixed.

15. The arrangement according to the invention, wherein
a mass ratio between the metered medication dose and the excipient deposit is selected to be in a range 1:20-20:1.

16. The arrangement according to the invention, wherein
a formulation of the dry powder medicament comprises, consists essentially of, or consists of inhalable powder particles having a mass median aerodynamic diameter from about 0.5 μm to about 5 μm.

17. The arrangement according to the invention, wherein
the excipient is selected from a group consisting of monosaccarides, disaccarides, polylactides, oligo- and polysaccarides, polyalcohols, polymers, salts or mixtures thereof.

18. The arrangement according to the invention, wherein
a formulation is selected of the at least one biologically acceptable excipient comprising large particles to at least 90% by mass.

19. The arrangement according to the invention, wherein
a selected formulation of the at least one biologically acceptable excipient comprises large particles bigger than 20 μm in size to at least 90% by mass.

20. A method of joining a metered, dry powder medication dose, comprising at least one active pharmaceutical ingredient, together with a dry powder excipient dose, comprising at least one biologically acceptable excipient, in a common dose container, said doses intended for delivery by use of a dry powder inhaler device, comprising the steps of
selecting a formulation of the dry powder medication dose consisting of inhalable powder particles having a mass median aerodynamic diameter within a range from about 0.5 μm to about 5 μm;
selecting a formulation of the at least one excipient comprising large particles to at least 90% by mass;
defining an appropriate mass ratio between a selected, therapeutically effective medication dose mass to be filled and the dose mass of the excipient, whereupon the corresponding excipient dose mass is calculated, and
metering and filling the selected, therapeutically effective medication dose and the calculated dose of the excipient, optionally by making one or more depositions per dose, into the common dose container.

21. The method according to embodiment 20, comprising the further step of
mixing the medication and the excipient doses after an individual metering operation but prior to depositing the metered doses into the dose container, whereby the doses are already at least partly mixed when deposited in said container.

22. The method according to embodiment 20, comprising the further step of agitating the dose container holding the metered doses using electrical or mechanical energy such that the doses inside the container become at least partly mixed.

23. The method according to embodiment 20 or 21, comprising the further step of agitating the dose container holding the metered doses using electrical or mechanical energy such that the doses inside the container become at least partly mixed.

24. The method according to embodiment 20, comprising the further step of
selecting the at least one excipient from a group consisting of monosaccarides, disaccarides, polylactides, oligo- and polysaccarides, polyalcohols, polymers, salts or mixtures thereof.

25. The method according to embodiment 20, comprising the further step of
selecting a formulation of the at least one biologically acceptable excipient comprising large particles bigger than 20 μm in size to at least 90% by mass.

26. The method according to embodiment 24, comprising the further step of
selecting a formulation of the at least one biologically acceptable excipient comprising large particles bigger than 20 μm in size to at least 90% by mass.

27. The method according to embodiment 20, comprising the further step of
defining the mass ratio between the medication dose and the excipient dose to be in a range 1:20-20:1.

28. The method according to embodiment 20, comprising the further step of
sealing the common dose container moisture-tight by using a high barrier seal.

29. A medical product comprising a dose container enclosing a dry powder medication dose, comprising at least one active pharmaceutical ingredient, and further enclosing a dry powder excipient dose, comprising at least one biologically acceptable excipient, said doses suitable for inhalation from the dose container by use of a dry powder inhaler device, wherein
the medication dose has a metered, therapeutically effective mass, said dose consisting of powder particles of a mass median aerodynamic diameter in a range from about 0.5 μm to about 5 μm;
the excipient dose has a metered mass calculated from a pre-defined mass-ratio relative the metered medication dose;
said medication and excipient doses, optionally split up in more deposits than one per dose in the common dose container, are arranged for a simultaneous release together upon an inhalation using the dry powder inhaler device.

30. The medical product according to embodiment 29, wherein the dose container holding the metered doses is agitated using electrical or mechanical energy such that the doses inside the container become at least partly mixed.

31. The medical product according to embodiment 29, wherein
a mass ratio between the metered medication dose and the excipient dose is selected to be in a range 1:20-20:1.

32. The medical product according to embodiment 29, wherein
the at least one excipient is selected from a group consisting of monosaccarides, disaccarides, polylactides, oligo- and polysaccarides, polyalcohols, polymers, salts or mixtures thereof.

33. The medical product according to embodiment 29, wherein
the at least one excipient consists to at least 90% by mass of particles 20 μm in size or bigger and optionally particles of sizes ranging between 0.5 and 10 μm.

34. A use of the medical product according to embodiment 29, wherein
an enhanced and consistent delivery of the active pharmaceutical ingredient of the medication dose is achieved and retention of the active pharmaceutical ingredient is minimized in any selected dry powder inhaler device where the medical product is applied.
   It will be understood by those skilled in the art that various modifications and changes may be made to the present invention without departing from the scope thereof, which is defined by the appended claims.

1. A method of improving an emitted dose of a metered dry powder medicament dose comprising at least one active pharmaceutical ingredient from a dry powder inhaler device, comprising:
placing a metered dose of at least one biologically acceptable excipient and the metered medicament dose in a common space of a dose container or internal aerosolization chamber in a manner such that the dose of the medicament and the dose of the excipient are aerosolized together by the inhaler device during an inhalation, whereby particles of the excipient dose set particles of the medicament dose free into a stream of inhalation air, thereby raising an emitted medicament dose mass and reducing retention of medicament particles inside the inhaler device, whereby the yield of the medicament dose increases due to the presence of the excipient dose as compared to both the absence of the excipient dose and the presence of the excipient dose in an amount of less than 20:1 wt/wt excipient/medicament, i.e. excipient's share of medicament and excipient doses taken together is less than 95%.

2. A composition comprising a metered dose of at least one biologically acceptable excipient and a metered dry powder medicament dose, both contained within a common space of a dose container or internal aerosolization chamber of a dry powder inhaler device in a manner such that the dose of the medicament and the dose of the excipient can be aerosolized together by the inhaler device during an inhalation, whereby particles of the excipient dose set particles of the medicament dose free into a stream of inhalation air, thereby raising an emitted medicament dose mass and reducing retention of medicament particles inside the inhaler device, whereby the yield of the medicament dose increases due to the presence of the excipient dose as compared to both the absence of the excipient dose and the presence of the excipient dose in an amount of less than 20:1 wt/wt excipient/medicament, i.e. excipient's share of medicament and excipient doses taken together is less than 95%.

3. A method for delivering a dose of a metered dry powder medicament comprising at least one active pharmaceutical ingredient from a dry powder inhaler device, comprising:
inhaling a metered dose of at least one biologically acceptable excipient and the metered medicament dose from a common space of a dose container or internal aerosolization chamber in a manner such that the dose of the medicament and the dose of the excipient are aerosolized together, whereby particles of the excipient dose set particles of the medicament dose free into a stream of inhalation air, thereby raising an emitted medicament dose mass and reducing retention of medicament particles inside the inhaler device, whereby the yield of the medicament dose increases due to the presence of the excipient dose as compared to both the absence of the excipient dose and the presence of the excipient dose in an amount of less than 20:1 wt/wt excipient/medicament, i.e. excipient's share of medicament and excipient doses taken together is less than 95%, with optional mixing of the excipient and medicament before and/or during inhalation.

4. An arrangement in a dry powder inhaler device for improving emitted dose of a metered dry powder medicament dose, comprising at least one active pharmaceutical ingredient, when said metered dose is delivered, wherein
a separately metered dose of at least one biologically acceptable excipient formulation is included with the metered medicament dose in a common space of a dose container or internal aerosolization chamber, said excipient dose having a pre-determined ratio of mass relative the metered medicament dose;
the dose of the medicament and the dose of the excipient are arranged in the dose container such that said doses become aerosolized together by the inhaler device during an inhalation, and
whereby particles of the excipient dose set particles of the medicament dose free into a stream of inhalation air, thereby raising an emitted medicament dose mass and reducing retention of medicament particles inside the inhaler device, whereby the yield of the medicament dose increases due to the presence of the excipient dose in the dose container.

5. A method of joining a metered, dry powder medication dose, comprising at least one active pharmaceutical ingredient, together with a dry powder excipient dose, comprising at least one biologically acceptable excipient, in a common dose container, comprising the steps of
selecting a formulation of the dry powder medication dose consisting of inhalable powder particles having a mass median aerodynamic diameter within a range from about 0.5 μm to about 5 μm;
selecting a formulation of the at least one excipient comprising large particles to at least 90% by mass;
defining an appropriate mass ratio between a selected, therapeutically effective medication dose mass to be filled and the dose mass of the excipient, whereupon the corresponding excipient dose mass is calculated, and
metering and filling the selected, therapeutically effective medication dose and the calculated dose of the excipient, optionally by making one or more depositions per dose, into the common dose container.

6. A medical product comprising a dose container enclosing a dry powder medication dose, comprising at least one active pharmaceutical ingredient, and further enclosing a dry powder excipient dose, comprising at least one biologically acceptable excipient, said doses suitable for inhalation from the dose container by use of a dry powder inhaler device, wherein
the medication dose has a metered, therapeutically effective mass, said dose consisting of powder particles of a mass median aerodynamic diameter in a range from about 0.5 μm to about 5 μm;
the excipient dose has a metered mass calculated from a pre-defined mass-ratio relative the metered medication dose;
said medication and excipient doses, optionally split up in more deposits than one per dose in the common dose container, are arranged for a simultaneous release together upon an inhalation using the dry powder inhaler device.
